(19) United States
(12) Patent Application Publication
(10) Pub. No.: US 2011/0098197 A1
(43) Pub. Date: Apr. 28, 2011

(54) Method for Specific Covalent Coupling of Antibody Using a Photoactivable Protein G Variant

(76) Inventors: Bong Hyun Chung, Daejeon (KR); Yong Won Jung, Daejeon (KR); Jeong Min Lee, Daejeon (KR)

(21) Appl. No.: 12/988,722

(22) PCT Filed: May 27, 2008
(86) PCT No.: PCT/KR08/02956

§ 371 Date: Jan. 10, 2011

(30) Foreign Application Priority Data
May 9, 2008 (KR) 10-2008-0043437

(51) Int. Cl. C40B 30/04; C07K 19/00; C12N 9/96; C07K 1/00; C40B 40/10; C40B 50/00

(52) U.S. Cl. 506/9; 530/350; 435/188; 530/402; 530/391.1; 506/18; 506/23

(57) ABSTRACT

The present invention relates to a protein G variant comprising a mutated Fc binding domain, which is prepared by substituting cysteine for specific residues of the Fc-binding domain of protein G, and a method for preparing the same. Further, the present invention relates to a protein G variant comprising a cysteine mutated Fc binding domain that is site-selectively modified with a UV cross-linker. Further, the present invention relates to a method for UV cross-linking the protein G variant with antibody. The present invention relates to a protein G variant-antibody conjugate that is prepared by the above method. Further, the present invention provides a method for screening or analyzing antigens using the conjugate. Furthermore, the present invention provides a biochip or biosensor fabricated by linking the protein G variant to the surface of a solid support, and a method for fabricating the same. In addition, the present invention provides a method for immobilizing antibodies and analyzing antigens using the biochip or biosensor.

### TECHNICAL FIELD

The present invention relates to a protein G variant comprising a mutated Fc binding domain, which is prepared by substituting cysteine for specific residues of the Fc-binding domain of protein G, and a method for preparing the same. Further, the present invention relates to a protein G variant comprising a cysteine mutated Fc binding domain that is site-selectively modified with a UV cross-linker.

Further, the present invention relates to a method for UV cross-linking the protein G variant with antibody. Further, the present invention relates to a protein G variant-antibody conjugate that is prepared by the above method. Further, the present invention provides a method for screening or analyzing antigens using the conjugate. Furthermore, the present invention provides a biochip or biosensor fabricated by linking the protein G variant to the surface of a solid support, and a method for fabricating the same. In addition, the present invention provides a method for immobilizing antibodies and analyzing antigens using the biochip or biosensor.

### BACKGROUND ART

The antibody has been widely used in medical studies concerning diagnosis and treatment of diseases as well as in biological analyses, because of its property of specifically binding to an antigen (Curr. Opin. Biotechnol. 12 (2001) 65-69, Curr. Opin. Chem. Biol. 5 (2001) 40-45). Recently, as an immunoassay, immunosensors have been developed which require the immobilization of an antibody on a solid support and which measure changes in current, resistance, mass, optical properties or the like (affinity biosensors. vol. 7: Techniques and protocols). Coupling of antibodies to solid surfaces or other biological/chemical molecules is a key step in the development of immune-based assays. The coupling allows not only antibody immobilization on solid surfaces but also site-selective tagging of antibodies with various materials.

Upon immobilization of antibodies on various solid surfaces or tagging of antibodies, it is very important to maintain the antigen-binding abilities of modified antibodies. Chemical immobilization techniques (Langumur, 1997, 13, 6485-6490) have been widely used because they show good reproducibility and a wide range of applications, due to their feature of allowing secure binding of proteins. Of these methods, covalent coupling of antibodies has been most widely used. However, since the amine groups of antibodies usually participate in chemical covalent bonding, the modified antibodies often lose their orientation on the solid support and thereby their activity to bind to antigens (Analyst 121, 29R-32R). Also, while antibodies can be coupled via carbohydrate chains or disulfide bridges, chemical treatments, such as strong oxidation and reduction, must be applied. Creating a coupling method that is site-selective and distant from antigen-binding sites with minimum antibody modifications remains a significant challenge in the development of assay systems.

### DISCLOSURE

### Technical Problem

Therefore, the present inventors have made an effort to develop a novel antibody coupling method. As a result, they prepared a protein G variant that is site-selectively tagged with a UV cross-linker benzophenone, and which offers a universal tool for site-selective and covalent coupling to the Fc region of antibodies. They found that the novel protein G variant allows the site-selective tagging or immobilization of antibodies and omits the need for chemical treatment of antibodies, thereby completing the present invention.

### Technical Solution

It is an object of the present invention to provide a protein G variant comprising a mutated Fc binding domain, which is prepared by substituting cysteine for one or more amino acids selected from the group consisting of 21Val, 29Ala, and 47Asp in the Fc binding domain of protein G.

More specifically, the object of the present invention is to provide a cysteine mutated protein G variant, represented by Tx-Ly-(cysteine-introducing protein G-Fc binding domain)n-Qz

(wherein T and Q are peptide tag proteins, L is a linker, x, y or z is each 0 or 1, and n is 1 to 3).

It is another object of the present invention to provide a protein G variant comprising a cysteine mutated Fc binding domain which is additionally modified with a UV cross-linker complex.

It is still another object of the present invention to provide a protein G variant of which three residues, 21Val, 29Ala and 47Asp are selectively tagged with the UV cross-linker.

It is still another object of the present invention to provide a method for preparing the protein G variant comprising a cysteine mutated Fc binding domain which is additionally modified with a UV cross-linker complex.

It is still another object of the present invention to provide a biochip or biosensor fabricated by linking the protein G variant to the surface of a solid support.

It is still another object of the present invention to provide a method for fabricating the biochip or biosensor.

It is still another object of the present invention to provide a method for inducing covalent immobilization of antibodies on a particle surface using the protein G variant.

It is still another object of the present invention to provide a method for analyzing antigens using the biochip or biosensor.

### DESCRIPTION OF DRAWINGS

FIG. 1 shows the crystal structure of an Fc-binding domain of protein G and Fc fragment complex, in which the residues modified with the UV cross-linker are indicated in blue;

FIG. 2 shows the peptide sequence of Fc-binding domain of protein G employed in the present invention and the protein G variants employed in the cross-linking;

FIG. 3 shows a synthetic method of benzophenone-ethylene glycol-maleimide (Benzophenone-EG-maleimide) used in the present invention;

FIG. 4 shows construction of a UV cross-linker-modified Fc binding domain and a schematic representation of cross-linking between the protein G variant and antibody;

FIG. 5 is a photograph of protein electrophoresis (SDS-PAGE) showing the protein G variants before and after modification with the UV cross-linker;

FIG. 6 is a photograph of protein electrophoresis (SDS-PAGE) showing UV cross-linking between the protein G variant and antibodies under reducing (A) and non-reducing (B) conditions;

FIG. 7 is a graph showing the changes in surface plasmon resonance signal after application of anti-CRP antibodies to a Neutravidin-immobilized biosensor and subsequent CRP interactions, in which anti-CRP antibodies were biotinylated using NHS-EZ-biotin or via UV cross-linking with biotin-protein G variant;

FIG. 8(A) is an image showing covalent immobilization of antibodies, in which glass surface was covered with free protein G, protein G variant and BSA, and then treated with Cy3-labeled antibody solution, following UV irradiation directly onto the glass surface, noncovalently bound proteins were removed by briefly washing with 10 mM NaOH, and fluorescence signals of covalently bound Cy3-antibodies were measured using a fluorescence scanner, and FIG. 8(B) is the result of SPR imaging and fluorescence measurements showing covalent immobilization of antibodies on the intended areas of gold and glass surfaces, in which gold and glass surfaces were covered with the protein G variant, followed by antibody treatment, and the surfaces were subsequently UV irradiated through a mask with 300 μm holes; and

FIG. 9 is a photograph showing protein electrophoresis (SDS-PAGE) of covalently and noncovalently bound antibodies on magnetic particles, after the magnetic particles covered with protein G or protein G variant were treated with antibodies, and subsequently UV irradiated.

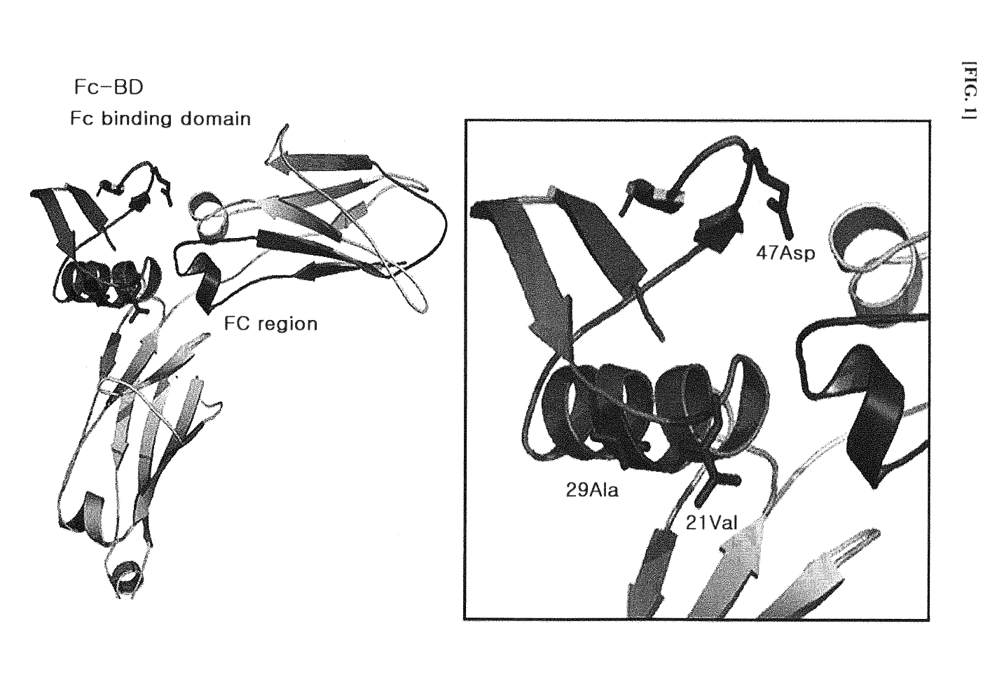

### BEST MODE

In accordance with one embodiment, the present invention provides a protein G variant comprising a mutated Fc binding domain, which is prepared by substituting cysteine for specific residues of the Fc-binding domain of protein G.

In one embodiment to achieve the object of the present invention, the present invention provides a cysteine mutated protein G variant, represented by Tx-Ly-(cysteine-introducing protein G-Fc binding domain)ₙ-Qz

(wherein T and Q are peptide tag proteins, L is a linker, x, y or z is each 0 or 1, and n is 1 to 3).

In the present invention, the origin of the protein G is not particularly limited, and the native protein G, an amino acid sequence of which is modified by deletion, addition, substitution or the like, may be suitably used for the purpose of the present invention, as long as it holds the ability to bind to an antibody. The protein G is preferably the Streptococcal protein G.

Fc domain is a region having a constant amino acid sequence of immunoglobulin or T cell receptor, and is not involved in binding with antigen. A higher order structure is formed by -S-S- loop with a set of amino acid residues, which are linked to each other by peptide bonds.

The Fc binding domain of protein G is known as a domain that binds to the Fc region of an antibody and constitutes the streptococcal protein G. Protein G is a bacterial cell wall protein isolated from group G streptococci. The domain has been known to bind to Fc and Fab regions of a mammalian antibody (J. Immuunol. Methods 1988, 112, 113-120). However, the protein G has been known to bind to the Fc region with

an affinity about 10 times higher than the Fab region. A DNA sequence of native protein G was analyzed and has been disclosed. A streptococcal protein G and streptococcal protein A are one of various proteins related to cell surface, which are found in Gram-positive bacteria, and have the property of binding to an immunoglobulin antibody. The streptococcal protein G variant, inter alia, is more useful than the streptococcal protein A, since the streptococcal protein G variant can bind to a wider range of mammalian antibodies, so as to be used as a suitable receptor for the antibodies.

The protein G comprises two or three Fc binding domains, denoted B1, B2 or C1, C2, C3, depending on the strain. The

protein G, an amino acid sequence of which may be modified by deletion, addition, substitution or the like, may be suitably used for the purpose of the present invention, as long as it holds the ability to bind to an antibody. The streptococcal protein G-B1 domain consists of three β-sheets and one α-helix, and the third β-sheet and α-helix in its C-terminal part are involved in binding to the antibody. Fc. As the amino acid sequences of B1 and B2 domains are compared to each other, there are differences in four sequences, but little difference between their structures. In one specific Example of the present invention, a B1 domain, in which ten amino acids were deleted at its N-terminal, was used. It was reported that even though a form of the B1 domain having ten amino acid residues deleted from the N-terminal side was used, there was no impact on the function of binding with an antibody (Biochem. J. (1990) 267, 171-177, J. mol. Biol (1994) 243, 906-918, Biochemistry (2000) 39, 6564-6571).

For the purpose of the present invention, the Fc binding domains, B1, B2 or C1, C2, C3 may be used, singly or in combination, to form multimers between homo-multimers or between hetero-multimers. In one Example of the present invention, the present inventors have used only antibody binding domains (B1, B2) of the gene of Streptococcal protein G.

The term “cysteine introducing protein G-Fc binding domain”, as used herein, refers to a protein G variant, which is prepared by substituting cysteine for a specific amino acid in the Fc binding domain of the protein G. The present invention provides a protein G variant comprising a cysteine mutated Fc binding domain. The amino acid sequence to be mutated to cysteine may be any region, as long as it

does not affect or hardly affects the antigen binding site. Preferably, one or more amino acids selected from the group consisting of 21Val, 29Ala, and 47Asp are substituted with cysteine. More preferably, one or more amino acids of 21 Val and 29Ala are substituted with cysteine. Most preferably, all of the amino acids are substituted with cysteine. In a specific Example of the present invention, provided is a protein G variant that is prepared by substituting cysteines for 21 Val and 29Ala.

The protein G variants of the present invention can be prepared by the methods widely known in the art, for example, a peptide synthesis method or a genetic engineering method, in particular, may be efficiently prepared by a genetic engineering method. The genetic engineering method is a method of expressing large amounts of the desired protein in a host cell such as E. coli by gene manipulation, and the related techniques are described in detail in disclosed documents (molecular biotechnology: Principle and Application of Recombinant DNA; ASM Press: 1994, J. chem. Technol. Biotechnol. 1993, 56, 3-13). Using the known techniques, a nucleic acid sequence encoding the protein G variant used in the present invention is contained in a suitable expression vector, and a suitable host cell is transformed with the expression vector, and cultured to prepare the protein G variants. More specifically, in the preferred Example of the present invention, the Fc binding domain was divided into two regions, and a gene for Fc binding domain with cysteine mutations at amino acids, 21 and 29 was prepared. Restriction enzyme sites were introduced at each end, and PCR was performed three times. The gene encoding a mutated Fc binding domain was obtained using two PCR products as a template, and then inserted into a vector, so as to obtain a cysteine mutated Fc binding domain.

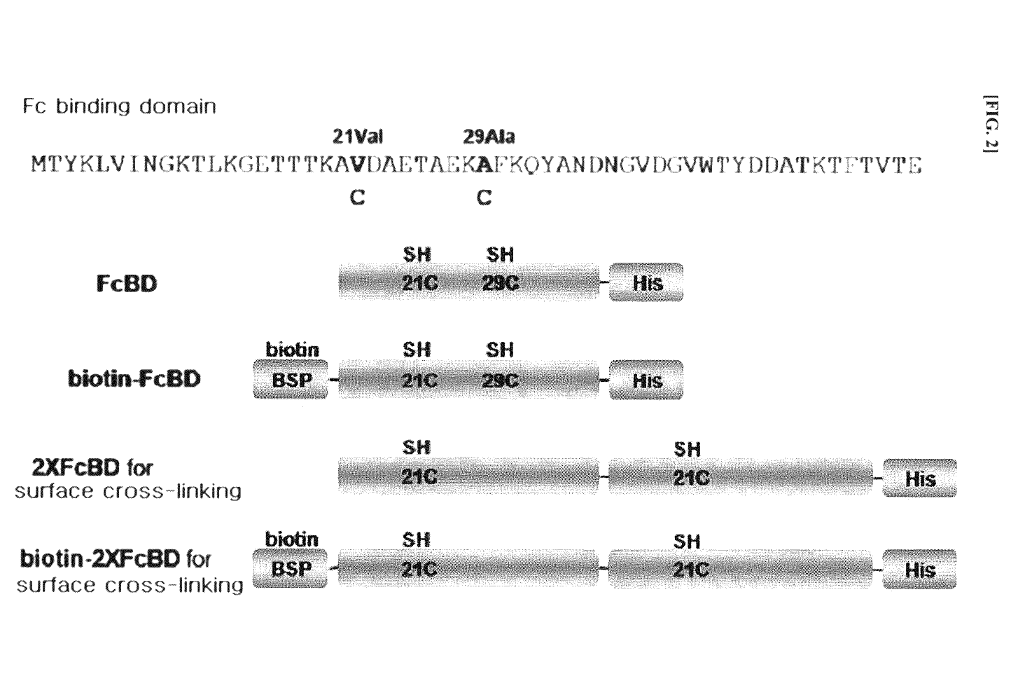

The T tag used in the present invention is not inserted inside of the protein G, and ensures the protein G adopts a proper orientation on attaching to a solid support. The T tag is not limited to its size or type, preferably any tag including biotin signaling peptide, histidine peptide (his), hemagglutinin (HA), Flag, gold binding peptide, and fluorescent proteins such as EGFP (enhanced GFP (Green Fluorescent Protein)), blue fluorescent proteins (EBFP (Enhanced Blue Fluorescent Protein), EBFP2, Azurite, mKalama1), cyan fluorescent proteins (ECFP (Enhanced Cyan Fluorescent Protein), Cerulean, CyPet), yellow fluorescent protein derivatives (YFP, Citrine, Venus, YPet), and BFP derivatives (Blue Fluorescent Protein derivatives). In addition, signal-amplifying enzyme such as alkaline phosphatase and peroxidase may be used. In a specific Example of the present invention, prepared was a cysteine mutated Fc binding domain variant comprising a biotinylation peptide sequence (FIG. 2).

The linker (L) used in the present invention functions to link the protein G variant with the T tag. In the protein G variant of the present invention, the tag (T) may be directly linked to the protein G by a covalent bond without the linker (L), or may be linked through the linker (L). The linker is a peptide having any sequence, which is inserted between the protein G and cysteine, and the number of amino acids of the linker is not limited. Preferably, the linker may be a peptide consisting of 2 to 10 amino acid residues.

In the present invention, a Q tag may be further included in the protein G variant, and it may be an additional tag for purification of the protein G variant. The Q tag may be further included at the C-terminal of the protein G. Preferably, the Q tag may be used as the tag for protein purification, and any known tag can be used without being limited thereto. Like the T tag, the Q tag is not limited in its size or type, and may be preferably any tag including biotin signaling peptide, histidine peptide (his), hemagglutinin (HA), Flag, gold binding peptide, and fluorescent proteins such as EGFP (enhanced GFP (Green Fluorescent Protein)), blue fluorescent proteins (EBFP (Enhanced Blue Fluorescent Protein), EBFP2, Azurite, mKalama1), cyan fluorescent proteins (ECFP (Enhanced Cyan Fluorescent Protein), Cerulean, CyPet), yellow fluorescent protein derivatives (YFP, Citrine, Venus, YPet), and BFP derivatives (Blue Fluorescent Protein derivatives). In addition, signal-amplifying enzyme such as alkaline phosphatase and peroxidase may be used.

According to another embodiment to achieve the objects of the present invention, a selectively reactive UV cross-linker complex is linked to a thiol group of the cysteine mutated protein G variant to prepare a protein G variant. The UV cross-linker complex consists of a UV cross-linker, a

side linker, and a reactive group, which are explained in detail herein below.

The term “UV cross-linker”, as used herein, refers to a substance that functions to link two substances with each other upon UV irradiation, in particular, a substance that functions to covalently link the Fc region of an antibody with the protein G variant of the present invention upon UV irradiation. Examples of the compound constituting the UV cross-linker may include benzophenone, aryl azide, and derivatives thereof. In a specific Example of the present invention, benzophenone was used as the UV cross-linker.

The “reactive group” is an active region that is introduced to react with cysteine of the cysteine mutated Fc binding domain for linkage with the UV cross-linker, and functions to link the UV cross-linker to the cysteine mutated protein G variant. Any reactive group may be used without limitation, as long as it is able to specifically react with the thiol group of cysteine. The reactive group that specifically reacts with the thiol group of cysteine is preferably maleimide.

The “side linker” is a compound that is introduced to link the UV cross-linker with the reactive group. The side linker that links the UV cross-linker with the reactive group, used in the present invention, functions to link the UV cross-linker to the reactive group that specifically reacts with the thiol group. The side linker is not limited in its type, and is preferably carbon chain or polyethylene glycol. More preferably, the side linker is ethylene glycol. In a specific Example of the present invention, an ethylene glycol (EG) side linker was used to improve in view of flexibility and hydrophilicity.

The “UV cross-linker complex” means a complex that is prepared by chemical linkage of three components. The UV cross-linker complex prepared by the above method has a reactive group capable of reacting with cysteine, for example, maleimide. Thus, it selectively reacts with the thiol group of the cysteine mutated protein G variant. In a specific Example of the present invention, prepared was a protein G variant tagged with benzophenone as the UV cross-linker at 21Val and 29Ala.

The UV cross-linker complex that specifically reacts with the thiol group of cysteine can covalently bind with antibodies upon UV irradiation, thereby performing various assays by the covalent coupling of antibodies.

Since the UV cross-linking between the protein G variant and antibodies in aqueous solution is an antibody-specific reaction, the composition of the aqueous solution is not limited, and other proteins may be included.

According to one embodiment, the present invention relates to a method for preparing the cysteine mutated protein G variant that is linked with a UV cross-linker complex. Specifically, the method for preparing the protein G variant according to the present invention comprises the steps of reducing the above described cysteine mutated protein G variant, removing the reducing agent, and reacting with the UV cross-linker complex, and further comprises the step of removing the unreacted UV cross-linker complex for purification.

According to another embodiment, the present invention provides a biochip or biosensor fabricated by linking the protein G variant to the surface of a solid support.

The solid support is used to provide successful UV-induced covalent immobilization of antibodies on the surface of the protein G variant-immobilized solid support. Any substrate may be used without limitation, as long as it is able to immobilize proteins. The antibody immobilization

may be performed on the surface of a thin film or particle. Preferably, the solid support may be selected from the group consisting of ceramics, glass, polymers, silicones, and metals, and more preferably, glass or gold. In a specific Example of the present invention, the present inventors performed the protein G immobilization and UV-induced covalent immobilization of antibodies on the surface of gold, glass slide and microparticles.

In still another embodiment, the present invention provides a method for fabricating the biochip or biosensor.

In still another embodiment, the present invention provides a method for inducing the covalent immobilization of antibodies on the surface of particles using the protein G variant. In a specific Example, the present inventors performed UV cross-linking in PBS buffer solution supplemented with BSA. Excess protein G variant can be removed by dialysis or gel-filtration.

In still another embodiment, the present invention relates to a method for analyzing antigens using the antibody immobilization method. The biochip or biosensor of the present invention is one kind of immunosensors, and thus any antigen analysis method using the widely known immunosensors may be applied thereto. Preferably, antigen analysis can be performed using the surface plasmon resonance-based biosensor.

### MODE FOR INVENTION

Hereinafter, the present invention will be described in detail with reference to Examples. However, these Examples are for illustrative purposes only, and the invention is not intended to be limited thereto.

### Example 1

### Preparation of Cysteine-Mutated Streptococcal Protein

FIG. 2 shows protein G variants employed in the present invention. First, in order to alter 21Val and 29Ala of the Fc binding domain into cysteine (FcBD; FIG. 2), PCR (polymerase chain reaction) was performed three times. The first PCR product contained the sequence encoding residues 1-27 of the Fc binding domain (FcBD), introducing 21Cys and an NdeI restriction enzyme site at the N-terminus. The second PCR reaction involved amplification of the sequence coding for amino acids 23-55 of the Fc binding domain, introducing 29Cys and an XhoI restriction enzyme site at the C-terminus. Both PCR products were used together as the template for the final PCR reaction with the sense primer of the first PCR reaction and the antisense primer of the second PCR reaction, so as to generate a gene for FcBD with cysteine mutations at 21Val and 29Ala. The final PCR product was inserted into the pET21a vector using two restriction enzymes, NdeI and XhoI.

RCRI: 5′ primer 1: sense
5-GGGAATTCCATATGACTTACAAACTTGTTATT-3
PCRI: 3′ primer 2: antisense
5-TTC TGC AGT TTC TGC GTC GCA TGC-3
RCRII: 5′ primer 1: sense
5-GCA GAA ACT GCA GAA AAA TGC TTC--3
PCRII: 3′ primer 2: antisense
5-GAGCTCGAGTTCAGTTACCGTAAAGGTCTTAGTC-3

To construct two proteins of 21Val mutated Fc-binding domain (2XFcBD; FIG. 2), PCR reaction was performed twice. The first reaction produced a PCR product encoding a 21Val mutated Fc binding domain with an N-terminal NdeI site and an extra seven amino acids at the C-terminus. The second PCR product contained extra eight amino acids at the N-terminus, a 21 Val mutated Fc binding domain, and a C-terminal XhoI site. Two PCR products were digested with each restriction enzyme. Digested products were ligated through their blunt ends, and inserted into pET21a.

RCRI: 5′ primer 1: sense
5-GGGAATTCCATATGACTTACAAACTTGTTATT-3
PCRI: 3′ primer 2: antisense
5-CGC ATC GAT CAC TTC TGG TTT TTC AGT TAC CGT AAA
GGT CTT-3
RCRII: 5′ primer 1: sense
5-TCT GAA TTA ACA CCA GCC GTG ACAACT TAC AAA CTT
GTT ATT AAT GG-3
PCRII: 3′ primer 2: antisense
5-GAGCTCGAGTTCAGTTACCGTAAAGGTCTTAGTC-3

To achieve biotinylation at the N-terminus, the biotinylation peptide sequence (GLNDIFEAQKIEWHE) was added to the N-terminus of protein G variant, and inserted into the vector pProExHTa. The proteins inserted into pET21a were expressed in E. coli BL21, and the biotinylated proteins inserted into the vector pProExHTa were expressed in AVB101 grown in the presence of 50 μM biotin. Protein expressions were induced at 25 by adding IPTG (isopropyl β-D-thiogalactopyranoside) at a final concentration of 1 mM. After 14 hrs, centrifugation was performed, and the obtained cell pellets were sonicated (Branson, Sonifier450, 3 KHz, 3 W, 5 min). A total protein solution was collected, and subjected to centrifugation to separate soluble and insoluble protein fractions. To purify each protein solution, a solution of disrupted cells in which the recombinant genes conjugated with hexahistidine were expressed, was loaded on a column packed with IDA excellulose. The recombinant proteins conjugated with histidine were eluted with an eluent (50 mM Tris-Cl, 0.5 M imidazole, 0.5 M NaCl, pH 8.0). For further purification of the obtained protein solution, the solution was loaded on a column packed with Q cellulose, and eluted with 1 M NaCl. Then, the eluted protein solution was dialyzed in PBS (phosphate-buffered Saline, pH 7.4) buffer solution containing 2 mM DTT.

### Example 2

### Synthesis of Maleimido-EG-Benzophenone

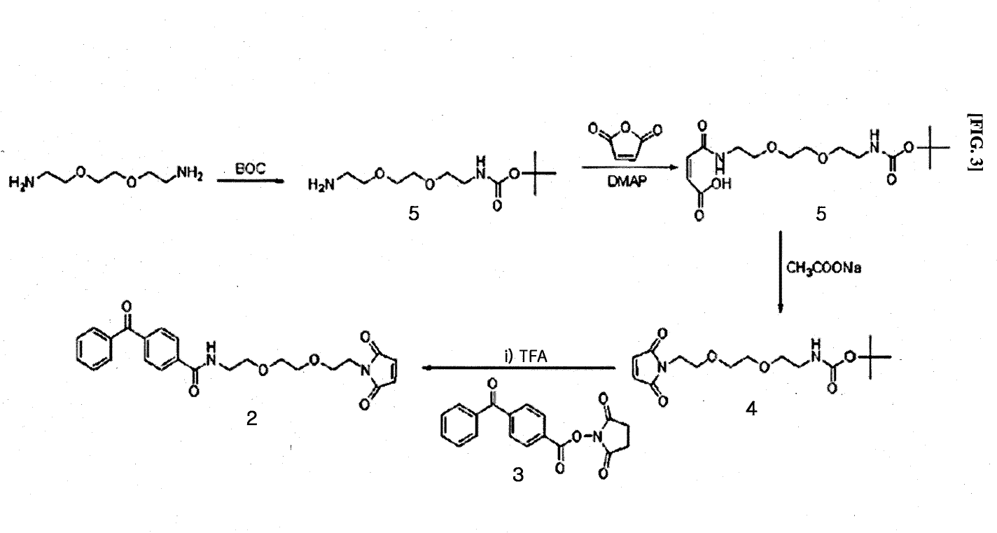

FIG. 3 shows the synthetic method for maleimido-EG-benzophenone 2. The synthetic method for Compound 4 is described in Korean Patent Application No. 10-2007-132998 contrived by the present inventors, and Compound 3 was synthesized in accordance with the published method (Biochemistry (1993) 32, 2741-2746). 0.3 g of Compound 4 was dissolved in a mixture of 10 mL of TFA (trifluoroacetic acid) and methylene chloride (1:1), and the solution was stirred at room temperature for 2 hrs. The organic solvent was removed under reduced pressure, and the process of dissolving the remaining material in methylene chloride and removing the solvent under reduced pressure was repeated three times to completely remove TFA. Approximately 0.14 g of deprotected compound 4 was obtained. To a mixture of 0.1 g (0.4 mmol) of deprotected 4 and 0.15 g (0.46 mmol) of Compound 3 in 20 mL of methylene chloride were added triethylamine (0.08 g, 0.8 mmol) and a catalytic amount of DMAP (dimethylaminopyridine) under nitrogen atmosphere. The reaction mixture was stirred at room temperature for 10 hrs. After the removal of solvent under reduced pressure, the residue was dissolved in 20 mL of methylene chloride and washed twice with distilled water. The resulting residue was purified through silica gel chromatography

(acetone/methylene chloride=1:3) to give a final yield of 90 mg of maleimido-EG-benzophenone 2 (47% yield).

### Example 3

### Preparation of Protein G Variant

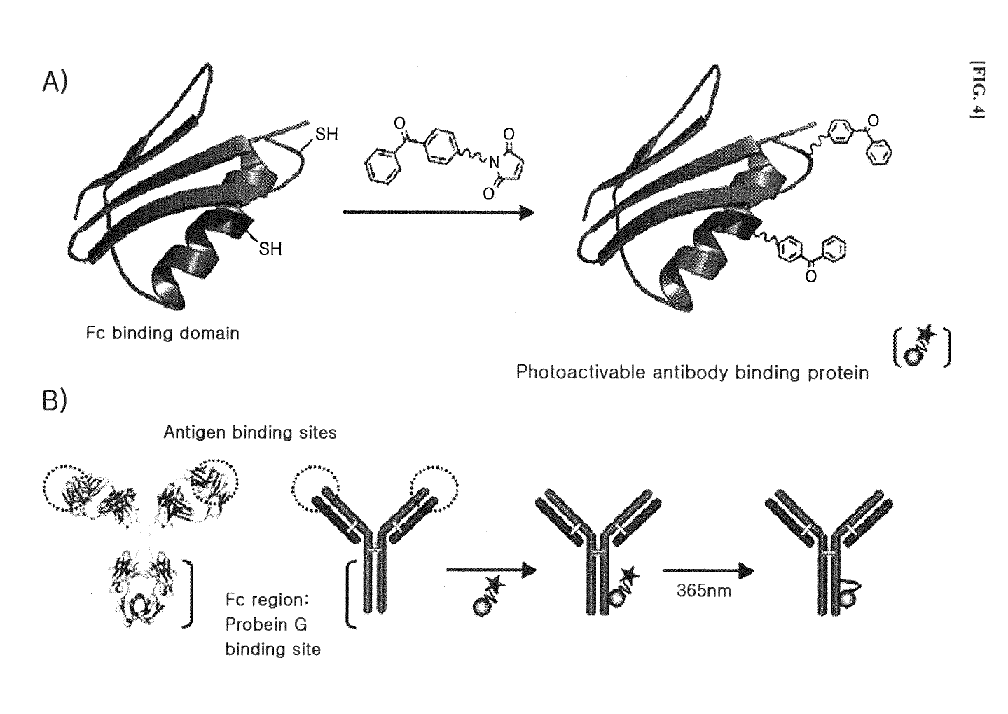

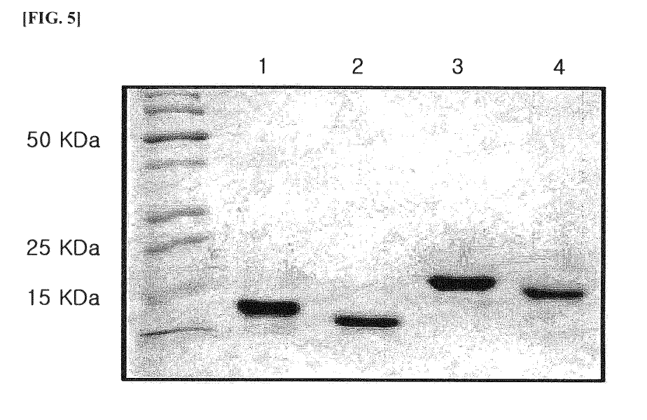

To prepare the protein G variant, the cysteine-mutated Fc binding domains were reacted with maleimido-EG-benzophenone 2 to modify the thiol group of cysteine with benzophenone (FIG. 4). The cysteine-mutated proteins were stored in buffer containing 2 mM DTT to maintain their reduced forms. Prior to reaction with Compound 2, DTT was removed by a desalting column. The resulting proteins were reacted with Compound 2 at room temperature for 1 hr. Excess Compound 2 was again removed by a desalting column. Successful benzophenone modification of cysteine mutated Fc binding domains was confirmed by SDS-PAGE, in which the proteins modified with a UV cross-linker benzophenone migrate differently from free proteins (FIG. 5).

The description of lanes in FIG. 5 is as follows;

Lane 1: cysteine-mutated Fc binding domain (FcBD),

Lane 2: FcBD reacted with Compound 2 (FcBD-BP),

Lane 3: two cysteine-mutated Fc binding domains (2XFcBD),

Lane 4: 2XFcBD (2XFcBD-BP) reacted with Compound 2

### Example 4

### UV Cross-Linking Between Antibody and Protein G Variant

UV cross-linking between the prepared protein G variants and antibodies was examined in solution.

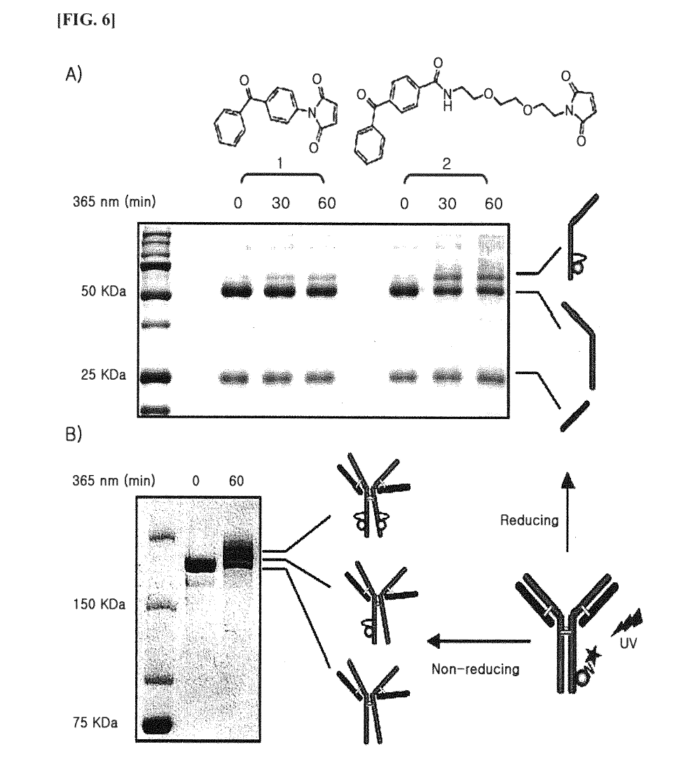

Specifically, about 5-fold protein G variant was incubated with 50 μg/mL antibodies in PBS buffer at room temperature for 30 min. Then, cross-linking was performed on ice for 30 min or 1 hr with UV light at 365 nm. SDS-PAGE analysis of cross-linked mixtures was performed under reducing conditions (FIG. 6A). Only heavy chains of antibodies were cross-linked to FcBD-BP, since the protein G variant targets only the Fc region of antibodies. When protein G variant was initially modified by commercially available maleimido-benzophenone 1, cross-linking was highly inefficient, indicating a critical role of the ethylene glycol linker between benzophenone and maleimide.

To investigate the overall cross-linking efficiency of intact antibodies to the protein G variant, the cross-linked product was analyzed under nonreducing conditions (FIG. 6B), where the intact form of the antibody was maintained disulfide bonds between heavy and light chains. It was found that since there were two protein G binding sites in the Fc region, more than 75% of antibodies are cross-linked to one or two FcBD-BP.

### Example 5

### Biotin Tagging of Antibody Using Protein G Variant

The Fc region of antibody was site-selectively biotinylated through the protein G variant, and the changes in surface plasmon resonance signal were measured using the surface plasmon resonance-based biosensor (SPR) in order to detect immobilization of the tagged antibody on solid surfaces.

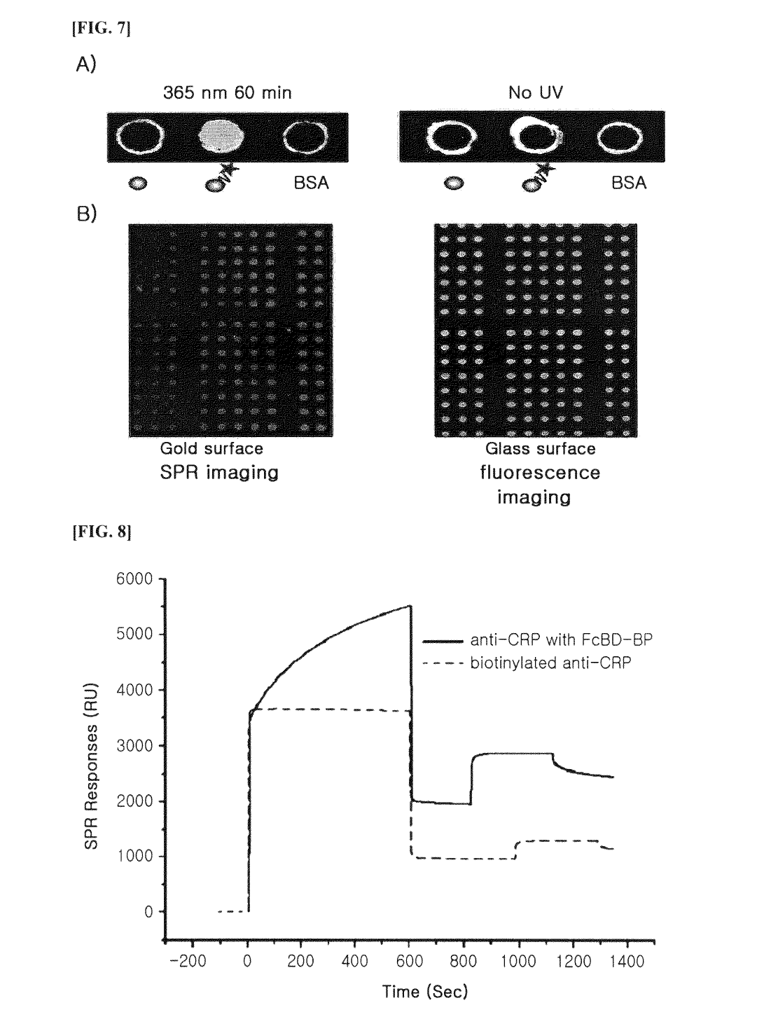

Specifically, anti-CRP (C-reactive protein) antibody was cross-linked with biotin-FcBD-BP by UV cross-linking method in the above described aqueous solution. Excess biotin-FcBD-BP was removed from the antibody by dialysis or gel-filtration. Anti-CRP antibody was also randomly biotinylated using NHS-EZ-biotin. The biotinylated anti-CRP antibodies were applied to a Neutravidin-immobilized biosensor, and subsequent CRP interactions were investigated using a SPR sensor (FIG. 7).

As a result, it was observed that the biotinylated antibodies were stably immobilized on the sensor surface through biotin-Neutravidin interaction. In comparison to randomly biotinylated antibody, biotin tagging via biotin-FcBD-BP induced stable immobilization of 1.5˜2 times as many anti-CRP antibodies on the chip surface. In addition, anti-CRP bound to biotin-FcBD-BP captures CRP proteins 3˜4-fold more efficiently than randomly biotinylated antibody. The results indicate that Fc-targeted antibody tagging via biotin-FcBD-BP provides more enhanced antibody immobilization than the known method.

### Example 6

### UV Cross-Linking Between Antibody and Protein G Variant for Immobilization on Solid Surface

UV-induced covalent immobilization of antibodies on solid surfaces was explored by using the protein G variant.

Specifically, glass or gold surface was covered with the protein G (2XFcBD-BP) or free protein G (2XFcBD) and BSA. The surface was treated with 50 ug/mL of PBS Cy3-labeled antibody solution for 30 min, and cross-linked with 365 nm UV light for 1 hr. Noncovalently bound proteins were removed by a brief wash with 10 mM NaOH for 1 min. Fluorescence signals of covalently bound Cy3-antibodies were measured using a fluorescence scanner.

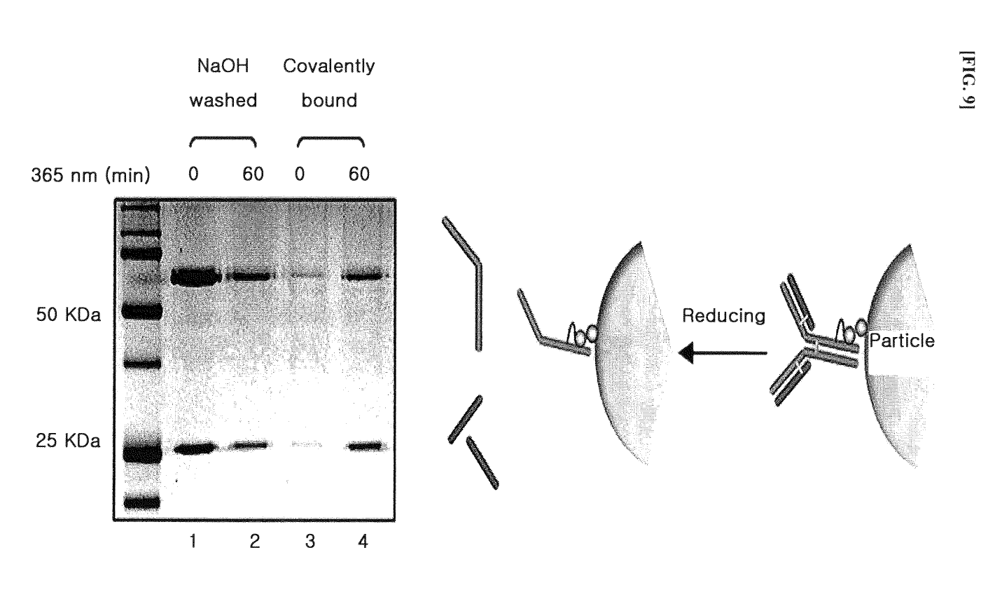

As a result, it was found that antibodies were specifically and covalently immobilized on the solid surface coated with the protein G variant (FIG. 8A).

Controlled covalent immobilization of antibodies was further investigated by using a mask with 300 μm spots. Glass or gold surface was covered with the protein G (2XFcBD-BP), followed by antibody treatment. The surfaces were subsequently irradiated with UV light through the mask. Antibody immobilization on the gold surface was examined by SPR imaging, and immobilization of Cy3-antibodies on the glass surface was examined using a fluorescence scanner. As a result, covalent immobilization of antibodies through 300 μm spots was only observed (FIG. 8B).

### Example 7

### UV Cross-Linking Between Antibody and Protein G Variant for Immobilization on Particle Surface

UV-induced covalent immobilization of antibodies on microparticle surfaces was explored by using the protein G variant.

Specifically, small magnetic particles containing carboxyl groups on the surface (DYNAL; Dynabeads MyOne™ Carboxylic Acid) were covered with the protein G variant (2XFcBD-BP) through NHS/EDC reaction. The modified magnetic particles were incubated with 50 μg/mL antibody. The mixture was irradiated with 365 nm UV light for 1 hr. Noncovalently bound proteins and covalently bound proteins were analyzed by SDS-PAGE.

As a result, in the absence of UV irradiation, most bound antibodies were released from the particles by 10 mM NaOH wash, whereas more than half of bound antibodies were covalently immobilized by UV cross-linking (FIG. 9).

The description of lanes in FIG. 9 is as follows;

Lane 1: antibodies released from particles (washed by 10 mM NaOH) without UV irradiation, after antibody treatment

Lane 2: antibodies released from particles (washed by 10 mM NaOH) with UV irradiation for 1 hr, after antibody treatment

Lane 3: covalently bound (10 mM NaOH wash resistant) antibodies without UV irradiation

Lane 3: covalently bound (10 mM NaOH wash resistant) antibodies after UV irradiation for 1 hr

### INDUSTRIAL APPLICABILITY

The protein G variants according to the present invention site-selectively capture antibodies and form covalent conjugates with captured antibodies upon UV irradiation. The protein G variants allow the site-selective tagging and immobilization of antibodies on the surface of biochip and biosensor with a highly preferred orientation.

What is claimed is:

1. A cysteine-mutated protein G variant represented by the following Formula:
Tx-Ly-(cysteine-introducing protein G-Fc binding domain)n-Qz
(wherein T and Q are peptide tag proteins, L is a linker, x, y or z is each 0 or 1, and n is 1 to 3).

2. The protein G variant according to claim 1, wherein the cysteine-mutated Fc binding domain is prepared by substituting cysteine for one or more amino acids selected from the group consisting of 21Val, 29Ala, and 47Asp.

3. The protein G variant according to claim 1 or 2, wherein a UV cross-linker complex selectively reacting with thiol group is additionally linked thereto.

4. The protein G variant according to claim 3, wherein the UV cross-linker complex consists of a UV cross-linker, a side linker, and a reactive group.

5. The protein G variant according to claim 4, wherein the UV cross-linker is benzophenone, aryl azide, or derivatives thereof.

6. The protein G variant according to claim 4, wherein the reactive group is maleimide or haloacetyl.

7. The protein G variant according to claim 4, wherein the side linker linking the reactive group with the UV cross-linker is carbon chain or ethylene glycol.

8. A protein G variant prepared by modifying one or more thiol groups in the cysteines of the protein G variant of claim 1 with UV cross-linker complex.

9. The protein G variant according to claim 1, wherein the T tag is one or more selected from the group consisting of biotin, hexa histidine peptide, hemagglutinin (HA), Flag, gold binding peptide, GFP (Green Fluorescent Protein), EGFP (enhanced GFP), BFP (Blue Fluorescent Protein), EBFP (Enhanced BFP), EBFP2, BFP derivatives, Azurite, mKalama1, ECFP (Enhanced Cyanide Fluorescent Protein), Cerulean, CyPet, YFP (Yellow Fluorescent Protein), Citrine, Venus, YPet, alkaline phosphatase, and peroxidase.

10. The protein G variant according to claim 1, wherein the Q tag is one or more selected from the group consisting of biotin, hexa histidine peptide, hemagglutinin (HA), Flag, gold binding peptide, GFP (Green Fluorescent Protein), EGFP (enhanced GFP), BFP (Blue Fluorescent Protein), EBFP (Enhanced BFP), EBFP2, BFP derivatives, Azurite, mKalama1, ECFP (Enhanced Cyanide Fluorescent Protein), Cerulean, CyPet, YFP (Yellow Fluorescent Protein), Citrine, Venus, YPet, alkaline phosphatase, and peroxidase.

11. A method for preparing the cysteine-mutated protein G variant according to claim 1.

12. The method for preparing the cysteine-mutated protein G variant according to claim 11, further comprising the step of separating/purifying the protein G variant after the linkage step.

13. A method for UV cross-linking the protein G variant of claim 3 with antibodies.

14. A protein G variant-antibody conjugate which is prepared by the method of claim 13.

15. A method for screening or analyzing antigens using the conjugate of claim 14.

16. A biochip or biosensor which is fabricated by linking the protein G variant of claim 1 to the surface of a solid support.

17. The biochip or biosensor according to claim 16, wherein the solid support is one selected from the group consisting of ceramics, glass, polymers, silicones, and metals.

18. The biochip or biosensor according to claim 16, wherein the biochip or biosensor is a gold thin film or gold nanoparticle.

19. The biochip or biosensor according to claim 16, wherein antibodies are additionally linked to the protein G variant immobilized on the surface of solid support.

20. A method for fabricating the biochip or biosensor of claim 16.

21. An antibody immobilization method using the protein G variant of claim 1 and UV light.

22. A method for analyzing antigens using the biochip or biosensor of claim 19.

23. The protein G variant according to claim 5, wherein the reactive group is maleimide or haloacetyl.

24. The protein G variant according to claim 5, wherein the side linker linking the reactive group with the UV cross-linker is carbon chain or ethylene glycol.

25. A method for preparing the cysteine-mutated protein G variant according to claim 2.

26. A method for preparing the cysteine-mutated protein G variant according to claim 3.

27. A biochip or biosensor which is fabricated by linking the protein G variant of claim 3 to the surface of a solid support.


SEQUENCE LISTING (4 sequences)

SEQ ID NO: 1 - DNA, 32 bp, Artificial Sequence
  sense primer for RCR1
gggaattccatatgacttacaaacttgttatt

SEQ ID NO: 2 - DNA, 24 bp, Artificial Sequence
  antisense primer for RCR1
ttctgcagtttctgcgtcgcatgc

SEQ ID NO: 3 - DNA, 24 bp, Artificial Sequence
  sense primer for RCR2
gcagaaactgcagaaaaatgcttc

SEQ ID NO: 4 - DNA, 34 bp, Artificial Sequence
  antisense primer for RCR2
gagctcgagttcagttaccgtaaaggtcttagtc